(19) United States
(12) Patent Application Publication
(10) Pub. No.: US 2026/0273304 A1
(43) Pub. Date: Sep. 17, 2026

(54) LASER IRRADIATION SYSTEM

(71) Applicant: Sang-Sul SEO, Seoul (KR)

(72) Inventor: Sang-Sul SEO, Seoul (KR)

(21) Appl. No.: 19/473,266

(22) PCT Filed: Jan. 17, 2024
(86) PCT No.: PCT/KR2024/000840

§ 371 Date: Oct. 7, 2025

(30) Foreign Application Priority Data
Apr. 13, 2023 (KR) 10-2023-0048799

(51) Int. Cl. A61N 5/06; A61N 5/067

(52) U.S. Cl. CPC A61N 5/0619; A61N 5/067; A61N 2005/0626; A61N 2005/0645; A61N 2005/0659; A61N 2005/0663

(57) ABSTRACT

Proposed is a laser irradiation system including a mobile application executed on a mobile terminal of a user, a management server for receiving a facial image of the user from the mobile application and analyzing the same so as to determine whether the user is a hypertensive patient, at least one laser irradiator in which a plurality of light-emitting elements for irradiating different parts of a body of the user in a lying position with laser light is arranged, and a controller for controlling the laser irradiator. The controller includes a communication unit for short-range data communication with the mobile application, an information storage unit in which acupuncture point information including acupuncture points for hypertension is stored, and a control unit driving and controlling the light-emitting elements corresponding to the acupuncture points for hypertension so as to irradiate the corresponding acupuncture points with laser light for a hypertensive patient.

### TECHNICAL FIELD

The present disclosure relates to a laser irradiation technology. More particularly, the present disclosure relates to a technology for performing laser irradiation by driving and controlling only some light-emitting elements.

### BACKGROUND ART

A semiconductor laser driving device is disclosed in Korean Patent Application No. 10-0866240. The semiconductor laser driving apparatus generates an optical pulse signal using a stable direct current supply and relaxation oscillation that is an inherent characteristic of laser, without using a conventional LED driving circuit, thereby driving a semiconductor laser and enabling an extension of a semiconductor laser lifespan. In addition, the semiconductor laser driving apparatus enables not only continuous driving of a laser beam but also an adjustment of a pulse peak value, a pulse width, and a pulse interval during pulse driving.

### DISCLOSURE

### Technical Problem

An objective of the present disclosure is to provide a method for automatically performing laser irradiation on a specific part of a body of a user.

### Technical Solution

According to an aspect of the present disclosure, there is provided a laser irradiation system including: a mobile application executed on a mobile terminal of a user ; a management server configured to determine whether the user is a hypertensive patient by receiving a facial image of the user from the mobile application and analyzing the facial image of the user; at least one laser irradiator in which a plurality of light-emitting elements for irradiating different parts of a body of the user in a lying position with laser light is arranged; and a controller configured to control the laser irradiator. In addition, the controller may include: a communication unit provided for short-range data communication with the mobile application; an information storage unit in which acupuncture point information including acupuncture points for hypertension is stored; and a control unit configured to drive and control the light-emitting elements corresponding to the acupuncture points for hypertension stored in the information storage unit so that corresponding acupuncture points are irradiated with laser light when data indicating that the user is a hypertensive patient is transmitted from the mobile application through the communication unit.

The acupuncture points for hypertension may include Renying (ST9) acupuncture point, Fengfu (GV16) acupuncture point, and Fengchi (GB20) acupuncture point. Furthermore, the control unit may be configured to drive and control the light-emitting elements corresponding to Renying (ST9) acupuncture point when a position of the user is a supine position, and may be configured to drive and control the light-emitting elements corresponding to Fengfu (GV16) acupuncture point and Fengchi (GB20) acupuncture point when the position of the user is a prone position.

The controller may further include a pressure pad configured to sense a downward pressure applied by the body of the user in the lying position, and the control unit may be configured to determine whether the position of the user is the supine position or the prone position according to a distribution of the downward pressure by the body of the user acquired through the pressure pad.

### Advantageous Effects

In the present disclosure, automatic laser irradiation based on a facial diagnosis is realized.

### DESCRIPTION OF DRAWINGS

FIG. 1 is a block diagram illustrating a a laser irradiation system according to an embodiment.

FIG. 2 is an example of mounting laser irradiators on a user's body.

### MODE FOR INVENTION

The above-described and additional aspects of the present disclosure will become more apparent through the preferred exemplary embodiments described with reference to the accompanying drawings. Hereinafter, the present disclosure will be described in detail so that those skilled in the art may easily understand and reproduce the present disclosure through such exemplary embodiments.

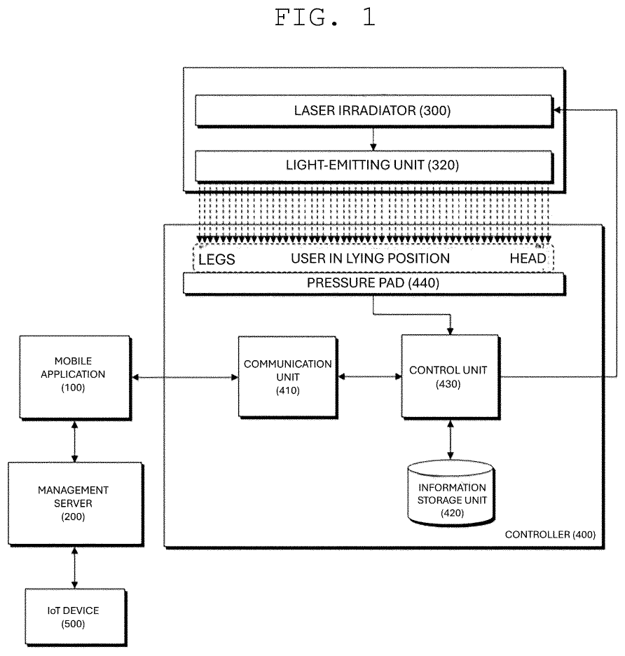

FIG. 1 is a block diagram illustrating a laser irradiation system according to an embodiment. A mobile application 100 is installed and executed on a mobile terminal of a user (for example, a smartphone), is capable of performing short-range data communication with a controller 400, and is capable of performing wireless data communication with a management server 200. The management server 200 is a server system including at least one server. Furthermore, the management server 200 may perform a function of determining whether the user is a hypertensive patient by analyzing a facial image of the user when the facial image of the user is transmitted from the mobile application 100 to the management server 200. At least one laser irradiator 300 is provided, and the laser irradiator 300 includes a light-emitting unit 320 and a driving unit 310. The light-emitting unit 320 includes a plurality of light-emitting elements for irradiating different points of a body of the user with laser light. The light-emitting element may be a diode, and is configured to emit light having a predetermined wavelength. For example, the light-emitting unit 320 is provided for a Low Level Laser Therapy (LLLT), and is configured to emit laser light having a wavelength in a range of 650 nm to 990 nm. In addition, the driving unit 310 is configured to drive only the light-emitting elements that are control targets to selectively emit light according to a driving control of the controller 400.

In an embodiment, the laser irradiator 300 is positioned on an upper side of a bed for the user to lie down or lie face down, and is configured to irradiate the body of the user in a lying position with laser light. That is, the laser irradiator 300 may be positioned on the upper side of the bed such that the laser irradiator 300 emits laser downward. In another embodiment, the laser irradiator 300 is a wearable device that is capable of being worn on the body of the user. For example, the laser irradiator 300 may include a laser irradiator worn on arms and a laser irradiator worn on legs, which may be further divided into a laser irradiator for the left arm, a laser irradiator for the right arm, a laser irradiator for the left leg, and a laser irradiator for the right leg.

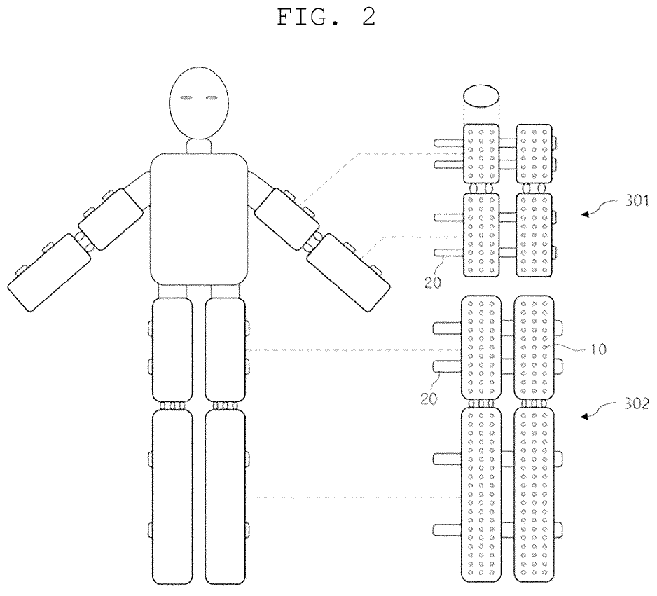

An example of this is illustrated in FIG. 2. A laser irradiator 301 worn arms and a laser irradiator 302 worn on legs are substantially identical except for different sizes for wearing, and include a plurality of light-emitting elements 10 arranged in rows and columns so as to irradiate different corresponding parts of the body with laser light. In addition, the laser irradiator 300 has a shape surrounding a body part so that both an inner side and an outer side of the body are capable of being irradiated with laser light, and may include a fixing means 20 such as a band for fixing the laser irradiator 300 while the laser irradiator 300 surrounds the body part. In addition, although not illustrated in FIG. 2, in addition to the laser irradiators for arms and legs, a laser irradiators worn on a neck portion and so on may be added.

The controller 400 is configured to control the laser irradiator 300 so that laser irradiation is performed on a specific part of the body of the user, and includes a communication unit 410, an information storage unit 420, and a control unit 430. The communication unit 410 is provided for short-range data communication with the mobile application 100, and a Bluetooth device may be given as an example. The information storage unit 420 is provided for data storage, and may be a flash memory or the like. In the information storage unit 420, acupuncture point information including acupuncture points for corresponding to each disease is stored and managed, and acupuncture points for hypertension is essentially included and managed in the acupuncture point information. In addition, in the information storage unit 420, information about the light-emitting elements corresponding to each acupuncture point may be stored and managed. In addition, the control unit 430 is a microprocessor, and is configured to control the laser irradiator 300 so that only some light-emitting elements of the light-emitting unit 320 selectively emit light.

Meanwhile, an IoT device 500 is mounted on a specific location, and is configured to perform a function of detecting barometric pressure, temperature, and humidity in real time and then providing the detected information to the management server 200. In addition, in this regard, the management server 200 is configured to perform a data management by receiving barometric pressure data, temperature data, and humidity data from the IoT device 500 and to provide these data to the mobile application 100 according to a request from the mobile application 100.

Hereinafter, an operation embodiment of the system illustrated in FIG. 1 will be described. The user manipulates the mobile application 100 and photographs the face of the user with a camera. Accordingly, the mobile application 100 acquires a facial image of the user and transmits the facial image to the management server 200. The management server 200 analyzes the facial image transmitted from the mobile application 100, determines whether the user is a hypertensive patient, and then transmits the determination result to the mobile application 100. A method of diagnosing hypertension through facial image analysis is already known. For example, the method is confirmed through a research result published in “Applied Sciences” under the title “A novel method in predicting hypertension using facial images” (published on Mar. 9, 2021).

When the mobile application 100 receives a result that the user is determined to be a hypertensive patient from the management server 200, the mobile application 100 transmits data indicating that the user is a hypertensive patient to the controller 400. Accordingly, the control unit 430 drives and controls the light-emitting elements corresponding to the acupuncture points for hypertension stored in the information storage unit 420, and irradiates the corresponding acupuncture points with laser light. For reference, the acupuncture points for relieving hypertension may be Renying (ST9) acupuncture point, Fengfu (GV16) acupuncture point, Fengchi (GB20) acupuncture point, Neiguan (PC6) acupuncture point, and so on as an example.

In addition, when the laser irradiator 300 is the same as illustrated in FIG. 2, it is possible to simultaneously irradiate a front surface and a rear surface of the body of the user with laser light. For example, it is possible to simultaneously irradiate the Renying (ST9) acupuncture point positioned on the front surface of the body and both the Fengfu (GV16) acupuncture point and the Fengchi (GB20) acupuncture point that are positioned on the rear surface of the body with laser light. However, when the laser irradiator 300 is positioned on the upper side of the bed and performs laser irradiation downward, the laser irradiation may only be performed on only the front surface of the body or only the rear surface of the body. Accordingly, when the user's position is a supine position, the control unit 430 drives and controls the light-emitting elements corresponding to the corresponding acupuncture points on the front surface of the body. When the user's position is a prone position, the control unit 430 drives and controls the light-emitting elements corresponding to the corresponding acupuncture points on the rear surface of the body. In the former case, the acupuncture point may be Renying (ST9) acupuncture point, and in the latter case, the acupuncture points may be the Fengfu (GV16) acupuncture point and the Fengchi (GB20) acupuncture point. In addition, whether the user's position is the supine position or the prone position may be confirmed through position information input by the user. For example, although not illustrated in FIG. 1, the controller 400 further includes a user interface for user input and output, and the user may input information on whether the user is lying down or lying face down through the user interface. Alternatively, the user may input the corresponding information through the mobile application 100 so that the corresponding information is transmitted to the controller 400.

In addition, the controller 400 may further include a pressure pad 440. The pressure pad 440 may be positioned on an upper portion of the bed, and may be configured such that pressure sensors are arranged inside the pressure pad 440 in a two-dimensional manner so that downward pressure generated by the user lying down on or lying face down on the pressure pad 440 is capable of being sensed, so that the pressure pad 440 is capable of outputting pressure sensing data when the pressure sensing is performed. In addition, the control unit 430 identifies a distribution of the downward pressure applied by the body of the user by analyzing the pressure sensing data input from the pressure pad 440, and determines whether the position of the user is the supine position or the prone position according to the identified distribution of the downward pressure. This is based on the fact that the distribution of body pressure in the supine position and the distribution of body pressure in the prone position are different. For example, in the lying down position, the pressure at the waist region (abdomen region) and the back of the knee region (knee region) is smaller than that of the pressure in the lying face down position, and in the lying face down posture, the pressure at the abdomen region and the knee region is larger than that of the pressure in the lying down position. According to the description described above, after automatically identifying whether the user's position is the supine position or the prone position, the laser irradiator 300 is capable of being driven and controlled.

Meanwhile, in the information storage unit 420, information on the light-emitting elements corresponding to each acupuncture point may be stored and managed together, and may be stored and managed separately for each body size. For example, information on light-emitting elements corresponding to each acupuncture point may be stored differently for each height. In addition, the control unit 430 receives body size information of the user from the mobile application 100 through the communication unit 410, and may drive and control the corresponding light-emitting elements according to the received body size information. In addition, information on the light-emitting elements corresponding to each body part may be stored and managed together in the information storage unit 420, and may also be stored and managed separately for each body size.

In addition, when the user's position is the supine position and the user is a gonarthritis patient, the controller 400 may drive and control the light-emitting elements corresponding to the knee region so that the knee region is irradiated with laser light. To this end, when the user is registered as a gonarthritis patient and the position of the user is determined to be the supine position by the controller 400, the mobile application 100 requests and receives barometric pressure data, temperature data, and humidity data detected by the IOT device 500 to the management server 200, and transmits the data to the controller 400. Here, the mobile application 100 requests and receives barometric pressure data, temperature data, and humidity data during a time the user was located at a specific place within a predetermined time (for example, 24 hours) prior to a current time. Since the mobile application 100 is capable of identifying a location of the user in real time by using a GPS resource of the mobile terminal, the mobile application 100 may request and acquire the barometric pressure data, the temperature data, and the humidity data during the time confirmed that the user is located at the specific place. Meanwhile, the specific place refers to a place specified by the user, may be a place in which environmental control by the user is difficult to be performed, and may be a workplace (office) of the user may be given as a representative example.

The control unit 430 compares the barometric pressure data, the temperature data, and the humidity data received from the mobile application 100 through the communication unit 410 with a preset appropriate barometric pressure range, a preset appropriate temperature range, and a preset appropriate humidity range, respectively, and determines whether any of the received data fall outside the respective appropriate ranges and identifies the duration of any such deviations, and subsequently determines an appropriate laser irradiation time for the knee region on the basis of the determination results. This considers that arthritis pain is greatly affected by humidity, temperature, and barometric pressure. Accordingly, when the barometric pressure, the temperature, and the humidity fall outside the respective appropriate ranges during the time the user stayed at the specific location, the control unit 430 determines the laser irradiation time by considering the duration of such deviation. In other words, the laser irradiation time is automatically increased according to the degree of deviation from the appropriate ranges, thereby facilitating more effective pain relief.

So far, the present disclosure has been described focusing on the preferred exemplary embodiments. A person skilled in the art to which the present disclosure pertains will understand that the present disclosure may be implemented in a modified form without departing from the essential characteristics of the present disclosure. Therefore, the disclosed exemplary embodiments should be considered from an illustrative rather than a restrictive perspective. The scope of the present disclosure is shown in the claims, not the above description, and all differences within the scope of equivalents are to be construed as being included in the present disclosure.

### DESCRIPTION OF REFERENCE NUMERALS

100: Mobile application 200: Management server
300: Laser irradiator 310: Driving unit
320: Light-emitting unit 400: Controller
410: Communication unit 420: Information storage unit
430: Control unit 440: Pressure pad
500: IOT device

1. A laser irradiation system comprising:
a mobile application executed on a mobile terminal of a user;
a management server configured to determine whether the user is a hypertensive patient by receiving a facial image of the user from the mobile application and analyzing the facial image of the user;
at least one laser irradiator in which a plurality of light-emitting elements for irradiating different parts of a body of the user in a lying position with laser light is arranged; and
a controller configured to control the laser irradiator,
wherein the controller comprises:
a communication unit provided for short-range data communication with the mobile application;
an information storage unit in which acupuncture point information including acupuncture points for hypertension is stored; and
a control unit configured to drive and control the light-emitting elements corresponding to the acupuncture points for hypertension stored in the information storage unit so that corresponding acupuncture points are irradiated with laser light when data indicating that the user is a hypertensive patient is transmitted from the mobile application through the communication unit.

2. The laser irradiation system of claim 1, wherein the acupuncture points for hypertension comprises a Renying (ST9) acupuncture point, a Fengfu (GV16) acupuncture point, and a Fengchi (GB20) acupuncture point, and
wherein the control unit is configured to drive and control the light-emitting elements corresponding to Renying (ST9) acupuncture point when a position of the user is a supine position, and is configured to drive and control the light-emitting elements corresponding to the Fengfu (GV16) acupuncture point and the Fengchi (GB20) acupuncture point when the position of the user is a prone position.

3. The laser irradiation system of claim 2, wherein the controller further comprises:
a pressure pad configured to sense a downward pressure applied by the body of the user in the lying position, and
wherein the control unit is configured to determine whether the position of the user is the supine position or the prone position according to a distribution of the downward pressure by the body of the user acquired through the pressure pad.

4. The laser irradiation system of claim 3, wherein the management server is configured to collect barometric pressure data, temperature data, and humidity data from an IoT device mounted in a specific place and to manage the barometric pressure data, the temperature data, and the humidity data,
wherein, when the user is registered as a gonarthritis patient and the position of the user is determined to be the supine position by the controller, the mobile application requests and receives barometric pressure data, temperature data, and humidity data during a time the user was located at the specific place within a predetermined time prior to a current time, and
wherein the control unit determines a laser irradiation time for a knee region of the user considering the barometric pressure data, the temperature data, and the humidity data transmitted from the mobile application, and then drives and controls the light-emitting elements corresponding to the knee region so that laser irradiation is performed for the determined laser irradiation time.
